(19) United States
(12) Patent Application Publication
(10) Pub. No.: US 2011/0015503 A1
(43) Pub. Date: Jan. 20, 2011

(54) MEDICAL APPARATUS FOR COLLECTING PATIENT ELECTROENCEPHALOGRAM (EEG) DATA

(75) Inventors: David Joffe, Boulder, CO (US); David Oakley, Boulder, CO (US)

Correspondence Address: PATTON BOGGS LLP, 1801 CALFORNIA STREET, SUITE 4900, DENVER, CO, 80202, US

(73) Assignee: WAVi, Boulder, CO (US)

(21) Appl. No.: 12/889,655

(22) Filed: Sep. 24, 2010

Related U.S. Application Data
(63) Continuation-in-part of application No. 12/567,249, filed on Sep. 25, 2009.
Application No. 12/567,249 is a continuation-in-part of application No. 12/505,185, filed on Jul. 17, 2009.

(51) Int. Cl. A61B 5/0476; A61B 5/11

(52) U.S. Cl. 600/301; 600/544

(57) ABSTRACT

The EEG Processing Unit comprises a semi-rigid framework which substantially conforms to the Patient's head and supports a set of electrodes in predetermined loci on the Patient's head to ensure proper electrode placement. The EEG Processing Unit includes automated connectivity determination apparatus which can use pressure-sensitive electrode placement ensuring proper contact with Patient's scalp and also automatically verifies electrode placement via measurements of electrode impedance through automated impedance checking. Voltages generated by the electrodes are amplified and filtered before being transmitted to an analysis platform, which can be a Physician's laptop computer system, either wirelessly or via a set of tethering wires. The EEG Processing Unit includes an automatic artifacting capability which identifies when there is sufficient clean data compiled in the testing session. This process automatically eliminates muscle- or other physical-artifact-related voltages. Clean data, which represents real brain voltages as opposed to muscle- or physical-artifact-related voltages, thereby are produced.

### CROSS-REFERENCE TO RELATED APPLICATIONS

This application is a continuation-in-part of U.S. patent application Ser. No. 12/567,249 filed on Sep. 29, 2009, which application is a continuation-in-part of U.S. patent application Ser. No. 12/505,185 filed on Jul. 17, 2009. This application is also related to an application filed concurrently herewith titled “Data Management Apparatus For Comparing Patient Data With Ailment Archetypes To Determine Correlation With Established Ailment Biomarkers” and an application filed concurrently herewith titled “Patient Data Management Apparatus For Comparing Patient EEG Data With Ailment Archetypes To Determine Correlation With Established Ailment Biomarkers.” The foregoing applications are hereby incorporated by reference to the same extent as though fully disclosed herein.

### FIELD OF THE INVENTION

This invention relates to medical apparatus and, in particular, to an apparatus for collecting patient electroencephalogram (EEG) data.

### BACKGROUND OF THE INVENTION

There are problems with the existing apparatus and methods used for collecting electroencephalogram data from a patient. These problems, as described herein, cause the data collection process to be time consuming, potentially inaccurate, and also costly due to the need for semi-skilled medical personnel to place the electrodes on the patient and execute the test process to obtain usable data for the treating Physician.

A routine clinical EEG recording typically lasts between 20 and 30 minutes (plus preparation time) and usually involves recording data that is obtained from scalp electrodes. In the conventional scalp EEG, the recording is obtained by placing a predetermined number of electrodes on the patient's scalp, in a set of predetermined locations, with a conductive gel or paste, usually after preparing the scalp area by light manual abrasion to reduce impedance due to dead skin cells. The electrodes are each attached to an individual wire, which serves to connect the electrodes to a data collection apparatus. Electrode locations and names are specified by the International 10-20 system for most clinical and research applications. This system ensures that the naming of electrodes is consistent. In most clinical applications, 19 recording electrodes (plus ground and system reference) are used.

Electrically, each electrode is connected, via its respective individual wire, to one input of a differential amplifier; and a common system reference lead is connected to the other input of each differential amplifier. These amplifiers amplify the voltage that is present between the active electrode and the system reference lead. In analog EEG, the signal is then filtered, and the EEG signal is output as a driving signal to cause the deflection of pens on a recorder as paper passes underneath the pens to draw waveforms representative of the time sequence of retrieved electroencephalogram data. Most EEG systems are now digital, and the amplified signal is digitized via an analog-to-digital converter, after being passed through an anti-aliasing filter. Analog-to-digital sampling typically occurs between 128 Hz and 512 Hz in clinical scalp EEG; sampling rates of up to 20 kHz are used in some research applications. The digital EEG signal is stored electronically and can be filtered for display. Typical settings for the high-pass filter and a low-pass filter are 0.5-1 Hz and 35-70 Hz, respectively. The high-pass filter typically filters out slow artifacts, such as electrogalvanic signals and movement artifacts, whereas the low-pass filter filters out high-frequency artifacts, such as electromyographic signals. An additional notch filter is typically used to remove 60 Hz artifacts caused by electrical power lines.

In this existing EEG paradigm, the test operator (typically specially trained medical personnel) must both place the electrodes in the proper location and also ensure that the electrodes have adequate contact with the skin to obtain usable voltage outputs for accurate readings. Marginal voltages and excessive artifacts are indicative of poor electrode placement as well as the long leads that carry the raw measured voltages to the data recorder. Furthermore, the accuracy of electrode placement is highly dependent on the skills of the medical personnel. Since the EEG data is complex and can only be properly interpreted to look for Ailments by a trained Physician, the determination of “good” EEG data is difficult to ascertain in the EEG test setting in real time.

Thus, there is presently no system that simplifies the electrode placement, ensures precise and secure electrode attachment, and reduces the effects of artifacts in the electroencephalogram data obtained using EEG tests.

### BRIEF SUMMARY OF THE INVENTION

The above-described problems are solved and a technical advance achieved by the present Medical Apparatus For Collecting Patient Electroencephalogram (EEG) Data (termed “EEG Processing Unit” herein), which enables the simple and efficient collection of accurate electroencephalogram data.

The EEG Processing Unit comprises a semi-rigid framework which substantially conforms to the head of the Patient. The framework (such as a mechanical lattice framework) supports a set of electrodes in predetermined loci on the Patient's head to ensure proper electrode placement. The EEG Processing Unit includes automated connectivity determination apparatus which can use pressure-sensitive electrode placement to ensure proper contact with the Patient's scalp and also automatically verifies the electrode placement via measurements of the electrode impedance through automated impedance checking. In addition, the EEG Processing Unit can include optional automated electrode movement or rotation apparatus to clean the skin of the Patient to optimize the electrode contact with the Patient's scalp as indicated by the measured impedance.

The voltages generated by the electrodes are amplified and filtered before being transmitted to an analysis platform, which can be a Physician's laptop computer system, either wirelessly or via a set of tethering wires. The EEG Processing Unit includes an automatic artifacting capability which identifies when there is sufficient clean data compiled in the testing session. This process automatically eliminates muscle or other physical artifact-related voltages. Clean data, which represents real brain voltages as opposed to muscle or physical artifact related voltages, thereby are produced. The automatic artifacting capability optionally includes an automatic Patient motion artifacting capability via an accelerometer that produces data indicative of Patient movement, which enhances the identification of accurate data.

Thus, the EEG Processing Unit provides EEG data collection capabilities heretofore unknown in the medical profession by enabling the simple and efficient collection of accurate electroencephalogram data via the use of the mechanical-lattice framework (exoskeleton) and its attached electrodes in combination with the associated automated impedance testing and artifacting.

### BRIEF DESCRIPTION OF THE DRAWINGS

FIG. 1 is a block diagram of the present EEG Processing Unit and an environment in which it is operational;

FIG. 2 is a block diagram illustrating an alternative embodiment of the EEG Processing Unit which makes use of a mechanical lattice framework in the EEG Processing Unit;

FIG. 3 is a block diagram illustrating an implementation of the EEG Transducer Placement System used in the EEG Processing Unit;

FIG. 4 is a block diagram illustrating an example electrode placement for gathering EEG data;

FIG. 5 illustrates a circuit diagram of the elements incorporated in the electrodes and the associated communications controller; and

FIG. 6 illustrates in flow diagram form the operation of the processor incorporated in the electrodes.

### DETAILED DESCRIPTION OF THE INVENTION

The EEG Processing Unit comprises a semi-rigid framework which substantially conforms to the head of the Patient. The framework supports a set of electrodes in predetermined loci on the Patient's head to ensure proper electrode placement. The EEG Processing Unit includes automated connectivity determination apparatus can use pressure-sensitive electrode placement to ensure proper contact with the Patient's scalp and also automatically verifies the electrode placement via measurements of the electrode impedance through automated impedance checking. In addition, the EEG Processing Unit can include optional automated electrode movement or rotation apparatus to clean the skin of the Patient to optimize the electrode contact with the Patient's scalp as indicated by the measured impedance.

The voltages generated by the electrodes are amplified and filtered before being transmitted to an analysis platform, which can be a Physician's laptop computer system, either wirelessly or via a set of tethering wires. The EEG Processing Unit includes an automatic artifacting capability which identifies when there is sufficient clean data compiled in the testing session. This process automatically eliminates muscle or other physical artifact-related voltages. Clean data, which represents real brain voltages as opposed to muscle or physical artifact-related voltages, are thereby produced. The automatic artifacting capability optionally includes an automatic Patient motion artifacting capability via an accelerometer that produces data indicative of Patient movement, which enhances the identification of accurate data.

### DEFINITIONS

Physician—is intended to include anyone who performs a diagnostic function, to review data about a Patient, and correlate that data with known ailments to provide the Patient with a diagnosis of their present state of health.

Ailment—is used in the general sense to represent any medical or psychological or physiological condition or problem that affects, or may in the future affect, a Patient, whether or not it is a threat to the Patient's life or health.

Electrode—as used herein, this term denotes the combination of a sensor element, which detects the EEG signals and converts them into electrical signals, which can optionally be colocated with the associated electronics which process the electrical signals as described herein.

### Source Of EEG Activity

The electrical activity of the brain can be described in spatial scales from either the currents that are generated within a single dendritic spine or the potentials that the EEG records from the Patient's scalp. Neurons, or nerve cells, are electrically active cells which are primarily responsible for carrying out the brain's functions. Neurons create action potentials, which are discrete electrical signals that travel down axons and cause the release of chemical neurotransmitters at the synapse, which is an area of near contact between two neurons. This neurotransmitter then activates a receptor in the dendrite or body of the neuron that is on the other side of the synapse, the post-synaptic neuron. The neurotransmitter, when combined with the receptor, typically causes an electrical current within the dendrite or body of the post-synaptic neuron. Thousands of post-synaptic currents from a single neuron's dendrites and body then sum up to cause the neuron to generate an action potential. This neuron then synapses on other neurons, and so on. A typical adult human EEG signal ranges from about 10 μV to 100 μV in amplitude when measured from the scalp.

An EEG reflects correlated synaptic activity caused by post-synaptic potentials of cortical neurons. The ionic currents involved in the generation of fast action potentials may not contribute greatly to the averaged field potentials representing the EEG. More specifically, the scalp electrical potentials that produce EEGs are generally thought to be caused by the extracellular ionic currents caused by dendritic electrical activity, whereas the fields producing magneto-encephalographic signals are associated with intracellular ionic currents.

The electric potentials generated by single neurons are far too small to be picked up by an EEG. Therefore, EEG activity always reflects the summation of the synchronous activity of thousands or millions of neurons that have similar spatial orientation, radial to the scalp. Currents that are tangential to the scalp are not picked up by the EEG. Therefore, the EEG benefits from the parallel, radial arrangement of apical dendrites in the cortex. Because voltage fields fall off with the fourth power of the radius, activity from deep sources is more difficult to detect than currents near the skull.

Scalp EEG activity shows oscillations at a variety of frequencies. Several of these oscillations have characteristic frequency ranges and spatial distributions and are associated with different states of brain functioning (e.g., waking and the various sleep stages). These oscillations represent synchronized activity over a network of neurons. The neuronal networks underlying some of these oscillations are understood, while many others are not.

### Clinical Use

A routine clinical EEG recording typically lasts between 20 and 30 minutes (plus preparation time) and usually involves recording from scalp electrodes which are manually placed in predetermined locations on the scalp of the Patient by the EEG test operator. A routine EEG is typically used in the following clinical circumstances, as ordered by a Physician to diagnose an Ailment and to:
distinguish epileptic seizures from other types of spells, such as psychogenic non-epileptic seizures, syncope (fainting), sub-cortical movement disorders and migraine variants;
differentiate “organic” encephalopathy or delirium from primary psychiatric syndromes such as catatonia;
serve as an adjunct test of brain death;
predict outcomes, in certain instances, in Patients with coma; and
determine whether to wean Patients off anti-epileptic medications.

Increasingly, EEG is also used as a diagnostic adjunct for:
learning and attention disorders;
dementing disorders; and
mood disorders.

Additionally, EEG, particularly digitized EEG, may be used to monitor certain procedures:
to monitor the depth of anesthesia;
as an indirect indicator of cerebral perfusion in carotid endarterectomy; and
to monitor amobarbital effect during the Wada test.

An EEG can also be used in intensive care units for brain function monitoring to:
monitor for non-convulsive seizures/non-convulsive status epilepticus;
monitor the effect of sedative/anesthesia in patients in medically induced coma (for treatment of refractory seizures or increased intracranial pressure); and
monitor for secondary brain damage in conditions such as subarachnoid hemorrhage (currently a research method).

In conventional scalp EEGs, the recording is obtained by the EEG test operator manually placing electrodes on the scalp with a conductive gel or paste, usually after manually preparing the scalp area by light abrasion to reduce impedance due to dead skin cells. Many systems typically use electrodes, each of which is attached to an individual wire. Some systems use caps or nets into which electrodes are embedded; this is particularly common when high-density arrays of electrodes are needed.

Electrode locations and names are specified by the International 10-20 system for most clinical and research applications and must be precisely followed by the EEG test operator in order to collect valid EEG data. This system ensures that the naming of electrodes is consistent across laboratories. In most clinical applications, 19 recording electrodes (plus ground and system reference) are used. A smaller number of electrodes are typically used when recording EEGs from neonates. Additional electrodes can be added to the standard set-up when a clinical or research application demands increased spatial resolution for a particular area of the brain.

Each electrode is connected to one input of a differential amplifier (one amplifier per pair of electrodes); a common system reference electrode is connected to the other input of each differential amplifier. These amplifiers amplify the voltage between the active electrode and the reference. In analog EEGs, the signal is then filtered, and the EEG signal is output as the deflection of pens as paper passes underneath. Most EEG systems are digital, and the amplified signal is digitized via an analog-to-digital converter, after being passed through an anti-aliasing filter. Analog-to-digital sampling typically occurs between 128 Hz and 512 Hz in clinical scalp EEGs; sampling rates of up to 20 kHz are used in some research applications.

The digital EEG signal is stored electronically and can be filtered for display. Typical settings for the high-pass filter and a low-pass filter are 0.5-1 Hz and 35-70 Hz, respectively. The high-pass filter typically filters out slow artifact, such as electrogalvanic signals and movement artifact, whereas the low-pass filter filters out high-frequency artifacts, such as electromyographic signals. An additional notch filter is typically used to remove artifact caused by electrical power lines (60 Hz in the United States and 50 Hz in many other countries).

### EEG Limitations

EEG measurements have several limitations. Most important is its poor spatial resolution. EEGs are most sensitive to a particular set of post-synaptic potentials: those which are generated in superficial layers of the cortex, on the crests of gyri directly abutting the skull and radial to the skull. Dendrites which are deeper in the cortex, inside sulci, in midline or deep structures (such as the cingulate gyrus or hippocampus), or producing currents which are tangential to the skull, have far less contribution to the EEG signal. The meninges, cerebrospinal fluid, and skull “smear” the EEG signal, obscuring its intracranial source.

The EEG is typically described in terms of (1) rhythmic activity and (2) transients. The rhythmic activity is divided into bands by frequency. To some degree, these frequency bands are a matter of nomenclature (i.e., any rhythmic activity between 8 Hz and 12 Hz can be described as “alpha”), but these designations arose because rhythmic activity within a certain frequency range was noted to have a certain distribution over the scalp or a certain biological significance. Frequency bands are usually extracted using spectral methods (for instance Welch) as implemented, for instance, in freely available EEG software such as EEGLAB. Most of the cerebral signal observed in the scalp EEG falls in the range of between 1 Hz and 50 Hz (activity below or above this range is likely to be artifactual, under standard clinical recording techniques, for example 0.5 Hz head motion or 60 Hz background noise can overwhelm the actual EEG signal).

### EEG Wave Patterns

### Delta Waves

Delta is the frequency range up to 4 Hz. It tends to be the highest in amplitude and the slowest waves. It is seen normally in adults in slow wave sleep. It is also seen normally in babies. It may occur focally with subcortical lesions and in general distribution with diffuse lesions, metabolic encephalopathy hydrocephalus, or deep midline lesions. It is usually most prominent frontally in adults (e.g., FIRDA—Frontal Intermittent Rhythmic Delta) and posteriorly in children (e.g., OIRDA—Occipital Intermittent Rhythmic Delta).

### Theta Waves

Theta is the frequency range from 4 Hz to 7 Hz. Theta is seen normally in young children. It may be seen in drowsiness or arousal in older children and adults; it can also be seen in meditation. Excess theta for age represents abnormal activity. It can be seen as a focal disturbance in focal subcortical lesions; it can be seen in generalized distribution in diffuse disorder, metabolic encephalopathy, deep midline disorders, or some instances of hydrocephalus. Alternatively, this range has been associated with reports of relaxed, meditative, and creative states.

### Alpha Waves

Alpha is the frequency range from 8 Hz to 12 Hz, and these waves are seen in the posterior regions of the head on both sides, being higher in amplitude on the dominant side. Activity in this EEG band is generated by closing the eyes and by relaxation. Alpha has been observed to attenuate with eye opening or mental exertion. This activity is now referred to as “posterior basic rhythm,” the “posterior dominant rhythm,” or the “posterior alpha rhythm.” The posterior basic rhythm is actually slower than 8 Hz in young children (therefore, technically in the theta range). In addition to the posterior basic rhythm, there are two other normal alpha rhythms that are typically discussed: the mu rhythm and a temporal “third rhythm.” Alpha can be abnormal; for example, an EEG that has diffuse alpha occurring in coma and is not responsive to external stimuli is referred to as “alpha coma.”

Sensorimotor Rhythm a/k/a Mu Rhythm

Mu rhythm is alpha-range activity that is seen over the sensorimotor cortex. It characteristically attenuates with movement of the contralateral arm (or mental imagery of movement of the contralateral arm).

### Beta Waves

Beta is usually defined as encompassing the frequency range from 12 Hz to about 30 Hz. It is seen usually on both sides in symmetrical distribution and is most evident frontally. Beta activity is closely linked to motor behavior and is generally attenuated during active movements. Low amplitude beta with multiple and varying frequencies is often associated with active, busy, or anxious thinking and active concentration. Rhythmic beta with a dominant set of frequencies is associated with various pathologies and drug effects. It may be absent or reduced in areas of cortical damage. It is the dominant rhythm in patients who are alert or anxious or who have their eyes open.

### Gamma Waves

Gamma is the frequency range between approximately 30 Hz and 100 Hz. Gamma rhythms are thought to represent binding of different populations of neurons together into a network for the purpose of carrying out a certain cognitive or motor function.

“Ultra-slow” or “near-DC” activity is recorded using DC amplifiers in some research contexts. It is not typically recorded in a clinical context because the signal at these frequencies is susceptible to a number of artifacts.

### Variants

Some features of the EEG are transient rather than rhythmic. Spikes and sharp waves may represent seizure activity or interictal activity in individuals with epilepsy or a predisposition toward epilepsy. Other transient features are normal: vertex waves and sleep spindles are transient events which are seen in normal sleep. It should also be noted that there are types of activity which are statistically uncommon but are not associated with dysfunction or disease. These are often referred to as “normal variants.” The mu rhythm is an example of a normal variant.

Background electroencephalography (EEG) produces predictable results in healthy, normally functioning individuals, but abnormal findings have been reported across a wide variety of ailments. The normal EEG varies by age. The neonatal EEG is quite different from the adult EEG. The EEG in childhood generally has slower frequency oscillations than the adult EEG. Patients with Alzheimer's disease, on the other hand, can be characterized by increased Delta and Theta and a slowing of the Alpha rhythm, for example, and patients with attention disorders by increased Theta corresponding to a decrease in Beta rhythms. The normal EEG also varies depending on state. The EEG is used along with other measurements (EOG, EMG) to define sleep stages in polysomnography. Stage I sleep (equivalent to drowsiness in some systems) appears on the EEG as drop-out of the posterior basic rhythm. There can be an increase in theta frequencies. Stage II sleep is characterized by sleep spindles—transient runs of rhythmic activity between 12 Hz and 14 Hz (sometimes referred to as the “sigma” band) that have a frontal-central maximum. Most of the activity in Stage II is in the 3 Hz to 6 Hz range. Stages III and IV sleep are defined by the presence of delta frequencies and are often referred to collectively as “slow-wave sleep.” Stages I-IV comprise non-REM (or “NREM”) sleep. The EEG in REM (rapid eye movement) sleep appears somewhat similar to the awake EEG.

An EEG measured on a Patient under general anesthesia depends on the type of anesthetic employed. With halogenated anesthetics, such as halothane or intravenous agents, such as propofol, a rapid (alpha or low beta), nonreactive EEG pattern is seen over most of the scalp, especially anteriorly (in some older terminology, this was known as a WAR (widespread anterior rapid) pattern), contrasted with a WAIS (widespread slow) pattern associated with high doses of opiates. Anesthetic effects on EEG signals are beginning to be understood at the level of drug actions on different kinds of synapses and the circuits that allow synchronized neuronal activity.

### Artifacts

### Biological Artifacts

Electrical signals detected along with biologically generated signals on the scalp by an EEG, but which have a non-cerebral origin, are called artifacts. EEG data is almost always contaminated by such artifacts. The amplitude of artifacts can be quite large relative to the size of amplitude of the cortical signals of interest. This is one of the reasons why it takes considerable experience to correctly interpret EEGs clinically. Some of the most common types of biological artifacts include:
Eye-induced artifacts (includes eye blinks and eye movements);
EKG (cardiac) artifacts;
EMG (muscle activation)-induced artifacts; and
Glossokinetic artifacts.

Eye-induced artifacts are caused by the potential difference between the cornea and retina, which is quite large compared to cerebral potentials. When the eye is completely still, this does not affect EEG. However, there are nearly always small or large reflexive eye movements, which generate a potential artifact which is picked up in the frontopolar and frontal leads. Involuntary eye movements, known as saccades, are caused by ocular muscles, which also generate electromyographic potentials. Purposeful or reflexive eye blinking also generates electromyographic potentials, but more importantly, there is reflexive movement of the eyeball during blinking which gives a characteristic artifactual appearance to the EEG.

Eyelid fluttering artifacts of a characteristic type were previously called Kappa rhythm (or Kappa waves). It is usually seen in the prefrontal leads, that is, just over the eyes. Sometimes they are seen with mental activity. They are usually in the Theta (4 Hz to 7 Hz) or Alpha (8 Hz to 13 Hz) ranges and are, in fact, noise in the EEG reading, and should not technically be called a rhythm or wave. Therefore, current usage in electroencephalography refers to the phenomenon as an eyelid fluttering artifact, rather than a Kappa rhythm (or wave).

EKG artifacts are quite common and can be mistaken for spike activity. Because of this, modern EEG acquisition commonly includes a one-channel EKG from the extremities. This also allows the EEG to identify cardiac arrhythmias that are an important differential diagnosis to syncope or other episodic/attack disorders. EKG artifacts can also be detected by means of signal processing algorithms which detect and then reject signals with repetitive characteristics typical of EKG.

Glossokinetic artifacts are caused by the potential difference between the base and the tip of the tongue. Minor tongue movements can contaminate the EEG, especially in Parkinson and tremor disorders.

### Environmental Artifacts

In addition to artifacts generated by the body, many artifacts originate from outside the body. Movement by the Patient, or even just settling of the electrodes, may cause electrode pops, i.e., spikes originating from a momentary change in the impedance of a given electrode. Poor grounding of the EEG electrodes can cause a significant 50 Hz or 60 Hz artifact, depending on the local power system's frequency. A third source of possible interference can be the presence of an IV drip; such devices can cause rhythmic, fast, low-voltage bursts, which may be confused for spikes.

### EEG Processing Unit

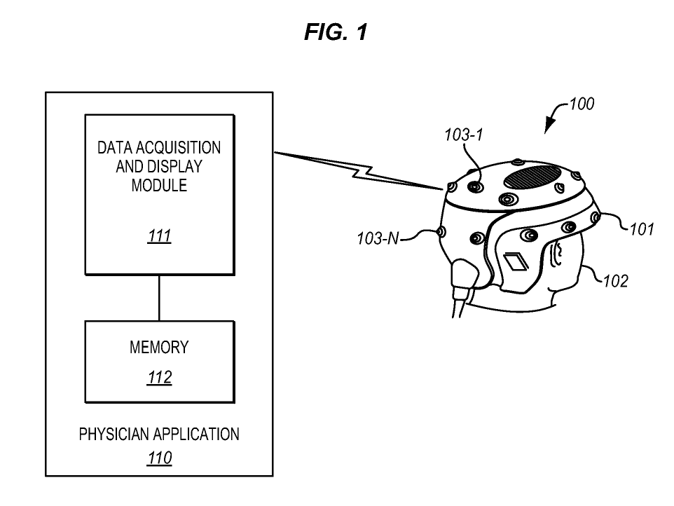

FIG. 1 is a block diagram of the present EEG Processing Unit and an environment in which it is operational. The EEG Processing Unit 100 includes “helmet-like” frame apparatus 101, which is typically semi-rigid in nature, conforms to the head of the Patient 102, and supports a set of electrodes 103-1 to 103-N, in predetermined loci, on the Patient's head to ensure proper electrode placement. Proper electrode placement is critical to the collection of accurate data to enable the Physician to obtain readings of the above-mentioned Waves and to distinguish anomalies in these Waves from normal patterns. In addition, associating electronics with the sensors in the EEG Processing Unit enables signal sampling and signal processing close to the source of the EEG signals so that the data that is transmitted for storage and review by the Physician is relatively noise-free before it leaves the EEG Processing Unit.

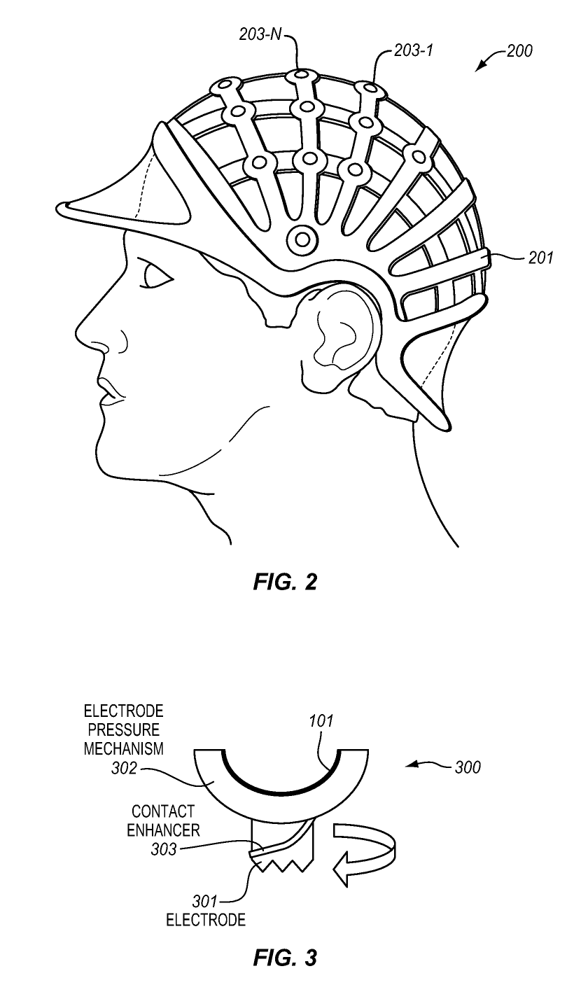

FIG. 2 is a block diagram illustrating an alternative embodiment of the EEG Processing Unit 100 which makes use of a framework 201 as an alternative to the “helmet” 101 design shown in FIG. 1. As with the “helmet” design, the framework 201 conforms to the head of the Patient and is a semi-rigid framework which supports a set of electrodes 203-1 to 203-N, in predetermined loci, on the Patient's head to ensure proper electrode placement.

FIG. 1 also illustrates a typical Physician Application 150 which is executing an application to receive and process EEG test data from and for a specific Patient. The Physician Application 110 shown in FIG. 1 includes the following components: Memory 112 and Data Acquisition and Display Module 113. Memory 112 stores the processed EEG data received from the EEG Processing Unit 100 via Data Acquisition and Display Module 113, which can generate displays of the various Waves which are described above.

### EEG Transducer Electrode Placement System

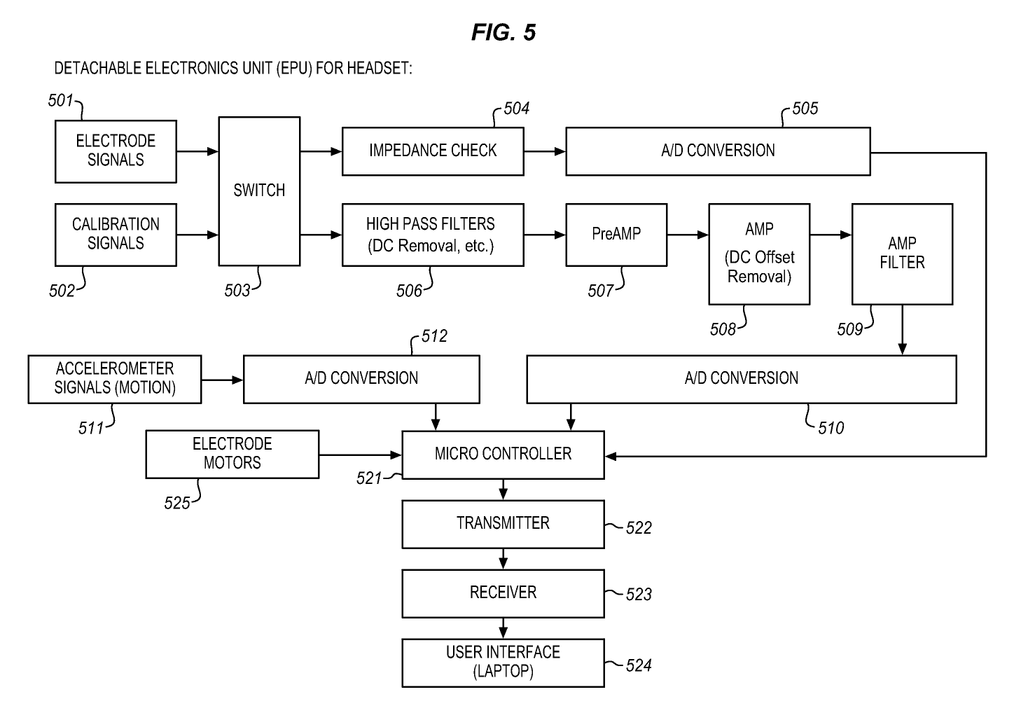

FIG. 3 is a block diagram illustrating an implementation of the EEG Electrode Placement System 300 used in the EEG Processing Unit 100. The EEG Electrodes 301 can optionally include the sensitive electronics, as shown in additional detail in FIG. 5, or the electronics can be housed in a separate unit. The EEG Electrodes 301 can include automated connectivity determination apparatus which uses pressure-sensitive electrode placement to ensure proper contact with the Patient's scalp and also automatically verifies the electrode placement via measurements of the electrode impedance through automated impedance checking. In particular, the EEG Electrode Placement System 300 includes an Electrode Pressure Mechanism 302 that, upon placement of the EEG Processing Unit 100 on the head of the Patient 102, is activated by the EEG test operator to apply pressure to the individual EEG Electrodes 301 which are attached to the framework 101 or 201 thereby to ensure secure contact of the EEG Electrode 301 with the scalp of the Patient 102. The Electrode Pressure Mechanism 302 consists of any of a spring mechanism, inflatable bladder(s), hydraulic plunger(s) and the like, which apply mechanical pressure to the “back side” of the EEG Electrodes 301 thereby to force them away from the interior surface of the framework 101 or 201 until the EEG Electrodes 301 come into firm contact with the scalp of the Patient 102.

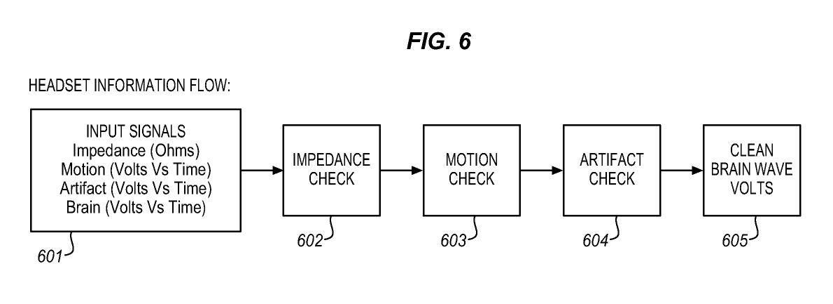

In addition, the EEG Processing Unit 100 can include optional automated Contact Enhancer Mechanism 303, with provides movement and/or rotation of the EEG Electrode 301 to clean the skin of the Patient 102 to optimize the electrode contact with the Patient's scalp as indicated by the measured impedance (described with respect to FIGS. 5 and 6).

### EEG Electrode Placement

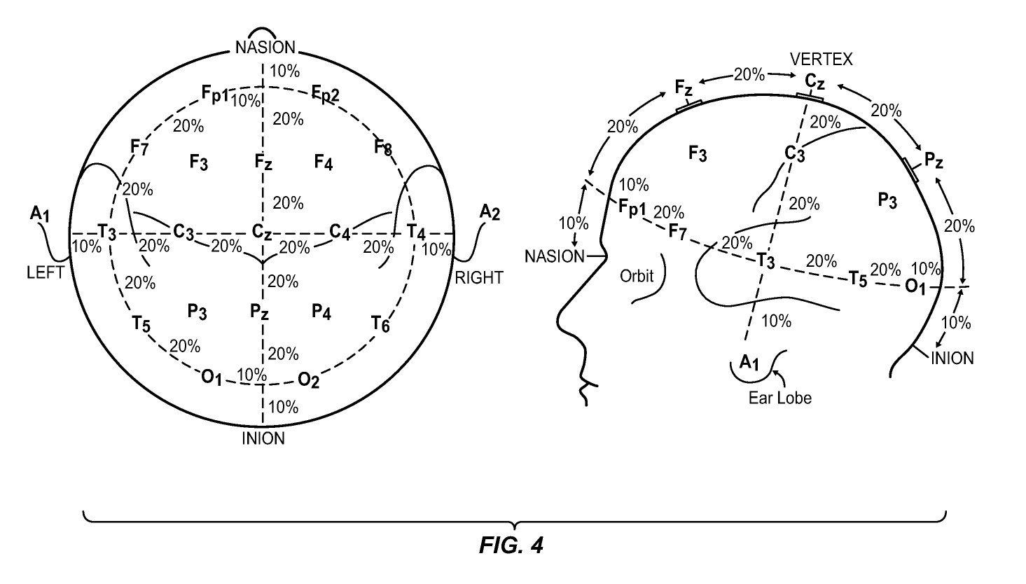

FIG. 4 is a block diagram illustrating an example electrode placement for gathering EEG data and represents electrode placement consistent with the International 10-20 EEG Classification System. Each electrode site has a letter to identify the lobe and a number or another letter to identify the hemisphere location. The letters C, F, Fₚ, O, P, and T stand for Central, Frontal, Frontal Pole, Occipital, Parietal, and Temporal locations of the brain, respectively. The even numbers refer to locations in the right hemisphere, the odd numbers refer to locations in the left hemisphere, and the letter “z” refers to an electrode placed on the midline. It is evident that, due to the number of the electrodes, the test operator must carefully associate each electrode with its predefined site on the Patient's head and ensure good physical contact of the electrode with the scalp before initiating the EEG test.

### EEG Electrode

FIG. 5 illustrates a circuit diagram of the elements associated with each of the electrodes (103-1 to 103-N and 203-1 to 203-N, which are collectively denoted as EEG electrode 500 in this Figure to describe a typical electrode) and the associated transmitter 522. This circuit can be detached and placed onto another headset in cases where differing head sizes is an issue.

The voltages generated by the EEG sensor 501 contained in the EEG Electrode 500 are amplified and filtered before being transmitted to an analysis platform, which can be a Physician's laptop computer system, either wirelessly or via a set of tethering wires. The EEG Processing Unit 100 includes automatic artifacting which identifies when there is sufficient clean data compiled in the testing session. This process eliminates muscle or other physical artifact-related voltages. Clean data, which represents real brain voltages as opposed to muscle or physical artifact related voltages, are thereby produced. The apparatus includes automatic motion artifacting via an accelerometer that produces data which enhances the identification of accurate data.

The data collected by the sensors can optionally be over sampled to enable a filter to effectively separate the signal from the noise. Over sampling is only performed on the pass-band information and not all of the data. One reason for over sampling only on the pass-band information is that it is not necessary to communicate all of the data but only the data in the pass-band. In traditional applications, in which the filtering was performed after the raw data was transmitted to a remotely located processor, all of the data was over sampled and sent over the communications channel. The use of over sampling and filtering in the EEG Processing Unit 100 reduces the bandwidth requirements of the data link and results in a cost savings over traditional systems. Furthermore, this architecture results in processing data with a signal-to-noise ratio that is lower than traditional systems. Consequently, the need for the use of conductive fluid on the sensor 501 can be reduced or even eliminated in some cases.

FIG. 5 is a block diagram illustrating the layout of various components of the EEG Electrode 500, which includes: EEG electrode signals 501; calibration signals 502; switch 503; impedance check 504; the filters 506, 509; the chain of amplifiers 507, 508; Analog to Digital (A/D) converters 505, 510, 512; optional accelerometer 511; optional electrode motor 525; and microcontroller 521, all located in or proximate to EEG Electrode 500 as shown in the Figures. The Analog-Digital Converters 505, 510, 512 and the microcontroller 521 can be part of the same electronic chip. One advantage of placing the microcontroller 521 in the EEG Electrode 500 assembly is that the data rate of the digital communications is kept to a minimum. In addition, the data processing task is distributed, simplifying the EEG Processing Unit 100 and, consequently, the cost.

FIG. 6 illustrates in flow diagram form the operation of the EEG

Processing Unit 100. At step 601, after the EEG Processing Unit 100 has been placed on the head of the Patient 102 and activated, the EEG Electrode 500 generates analog electrode signals 501 which contain multiple components: EEG signals, artifacts, and impedance measurements. The EEG voltages in electrode signals 501 can be replaced by calibration signals 502 generated by signal generators. Test waveforms are generated in software and then output as calibration signals 502, which are artificial representations of standard EEG, in both shape and voltage amplitudes, for the purpose of calibration and testing. In addition, accelerometer 511 generates motion signals indicative of the movement of the framework in three dimensions. A number of data processing steps operate on the EEG data to produce processed EEG data. In particular, at step 602, impedance measurement device 504 measures the impedance of the EEG sensor 500 which is indicative of the attachment of the EEG sensor 500 to the scalp of the Patient 102. Impedance is measured by applying a small AC voltage between each scalp electrode and the ground electrode and measuring the resultant peak-peak voltage. The results of this test are processed by A/D Converter 505 and transmitted by the microcontroller 521 to the user interface 524 for display via transmitter 522 and receiver 523 to enable the test operator to determine whether to proceed with the data collection process or readjust the EEG sensors 500. Alternatively, an automated electrode fit process can be executed, where the impedance values are fed to the microcontroller 521, which forwards these values (or other control signals) to the associated electrode positioning motors 525. The electrode positioning motor voltage and/or current readings are returned to the microcontroller 521; and if the measured impedance value was low, the electrode positioning motor 525 (a servo or stepping type of motor) moves to reposition the electrode on the scalp. The adjustment cycle continues until the specified impedance value is reached. Alternatively, inward motion of the electrode onto the scalp creates pressure; and the sensed electrode positioning motor drive current or voltage is monitored until the pressure cutoff value is reached, as indicated by the measured electrode positioning motor drive current or voltage.

At step 603, the presence of motion is determined by accelerometer 511 generating signals indicative of three-dimensional motion. The accelerometer 511 output is processed by A/D Converter 512 and transmitted by the microcontroller 521 to the user interface 524 for display via transmitter 522 and receiver 523 to enable the test operator to determine whether to proceed with the data collection process or give further instructions to the patient in cases where patient movement is interfering with the collection of biological brain-voltage signals. This information can also be used in post-hoc data analysis to either accept or reject a segment of data, or to recover the biologically generated portion of the measured EEG signal using algorithms that automatically subtract movement from data such as Independent Component Analysis (ICA) or related methods.

At step 604, low frequency components of possibly artifactual origin in the EEG data are processed by transmitting these analog signals via switch 503 to high pass filter 506 to remove DC components of the EEG data and out-of-band signals. Pre-Amplifier 507 and Amplifier 508 increase the magnitude of the EEG data signals, and these then are filtered by Amplifier Filter 509 before being converted to digital signals by A/D Converter 510. This processing is supplemented by software in microcontroller 521 where the EEG data then is processed by artifact-removal software to remove artifacts (e.g., electrical signals from muscle movement) to ensure that proper data was collected. Artifact detection serves three purposes: the first is to allow the administrator to instruct the patient when muscle-related artifacts are overwhelming the signal (for example, excessive eye movement or muscle tension); the second is to inform the administrator when enough clean data has been obtained and test is complete; and the third is for post-hoc data analysis which may include identification of clean epics or the cleaning of contaminated epics. The microcontroller 521 automatically determines whether the data is of adequate quality for transmission to the user interface 524 for display via transmitter 522 and receiver 523. The processed EEG data 605 (clean brain wave voltages) then is received by the Physician Application 110 where it is stored in memory 112 for later display by the Physician for analysis and diagnosis.

In response to receiving sets of EEG data relating to a Patient, a data selection physician interface screen can be displayed that allows for the sets of EEG data to be displayed in a raw data format, a topographic format, a trend analysis format, a spectral power format, a statistical characterization format, and/or the like. The data selection physician interface screen allows the Physician to select a desired display format and change between display formats through the use of radio buttons, drop-down menus, or other selection vehicles. In some cases, the data selection physician interface screen allows the Physician to select a portion of the data collected which is analyzed and/or displayed. For example, if a large amount of EEG data is collected under a variety of test conditions, the Physician could select the portion of the EEG data for analysis that is desired by the Physician.

### SUMMARY

The EEG Processing Unit comprises a semi-rigid framework which substantially conforms to the head of the Patient. The framework (such as a mechanical lattice framework) supports a set of electrodes in predetermined loci on the Patient's head to ensure proper electrode placement. The EEG Processing Unit includes automated connectivity determination apparatus which can use pressure-sensitive electrode placement to ensure proper contact with the Patient's scalp and also automatically verifies the electrode placement via measurements of the electrode impedance through automated impedance checking. In addition, the EEG Processing Unit can include optional automated electrode movement or rotation apparatus to clean the skin of the Patient to optimize the electrode contact with the Patient's scalp as indicated by the measured impedance.

What is claimed as new and desired to be protected by Letters Patent of the United States is:

1. An EEG Processing Unit for collecting EEG sensor data from a subject for forwarding to a data processing system, comprising:
a plurality of EEG sensors, responsive to the presence of EEG signals for generating electrical signals representative of said EEG signals;
frame having said plurality of EEG sensors mounted thereon in predetermined locations, for placement on a Patient's head thereby to site each of said plurality of EEG sensors at a corresponding predetermined location on a surface of the Patient's head to detect said EEG signals; and
signal processor, mounted on said frame, for processing said electrical signals representative of said EEG signals to remove artifacts therefrom.

2. The EEG Processing Unit of claim 1, further comprising:
impedance measurement for automatically performing measurements indicative of contact of said EEG sensors to said head of said Patient.

3. The EEG Processing Unit of claim 1, further comprising:
a test signal generator for executing a test to ascertain quality of signals being produced by said EEG Processing Unit.

4. The EEG Processing Unit of claim 1 wherein said signal processor comprises:
artifacting processor for processing said electrical signals produced by said EEG sensors to eliminate artifacts from said electrical signals to produce processed electrical signals.

5. The EEG Processing Unit of claim 4 wherein said signal processor further comprises:
data validation process for determining that said processed electrical signals are of sufficient quality to be output.

6. The EEG Processing Unit of claim 4, further comprising:
an accelerometer, attached to said frame and responsive to movement of said frame in three dimensions, for generating a motion signal representative of a direction and magnitude of said three-dimensional motion.

7. The EEG Processing Unit of claim 6 wherein said signal processor further comprises:
motion artifacting process, responsive to said motion signal, for processing said electrical signals to one of: remove motion artifacts from said electrical signals, and discard said electrical signals in the presence of movement of said frame.

8. The EEG Processing Unit of claim 1 wherein said frame comprises:
a semi rigid framework formed to substantially conform to the head of the Patient.

9. The EEG Processing Unit of claim 8, further comprising:
expansion mechanism connected to said frame for applying pressure to said plurality of EEG sensors to force said EEG sensors against the exterior surface of said Patient's head.

10. The EEG Processing Unit of claim 9, further comprising:
a contact enhancer mechanism for providing movement and/or rotation of said EEG sensors to enhance contact between said EEG sensors and the scalp of said Patient.

11. A method of collecting EEG sensor data from a Patient for forwarding to a data processing system, comprising:
generating, via a plurality of EEG sensors which are responsive to the presence of EEG signals, electrical signals representative of said EEG signals;
placing a frame having said plurality of EEG sensors mounted thereon in predetermined locations, on a Patient's head thereby to site each of said plurality of EEG sensors at a corresponding predetermined location on a surface of the Patient's head to detect said EEG signals; and
processing, in a processor mounted on said frame, said electrical signals representative of said EEG signals to remove artifacts therefrom.

12. The method of collecting EEG sensor data of claim 11, further comprising:
automatically performing measurements indicative of contact of said EEG sensors to said head of said Patient.

13. The method of collecting EEG sensor data of claim 11, further comprising:
executing a test to ascertain quality of signals being produced by said EEG Processing Unit.

14. The method of collecting EEG sensor data of claim 11 wherein said step of processing comprises:
processing said electrical signals produced by said EEG sensors to eliminate artifacts from said electrical signals to produce processed electrical signals.

15. The method of collecting EEG sensor data of claim 14 wherein said step of processing further comprises:
determining that said processed electrical signals are of sufficient quality to be output.

16. The method of collecting EEG sensor data of claim 4, further comprising:
generating, via an accelerometer which is attached to said frame and responsive to movement of said frame in three dimensions, a motion signal representative of a direction and magnitude of said three-dimensional motion.

17. The method of collecting EEG sensor data of claim 16 wherein said step of processing further comprises:
processing, in response to said motion signal, said electrical signals to one of: remove motion artifacts from said electrical signals, and discard said electrical signals in the presence of movement of said frame.

18. The method of collecting EEG sensor data of claim 11 wherein said step of placing comprises:
using a semi-rigid framework formed to substantially conform to the head of the Patient.

19. The method of collecting EEG sensor data of claim 18, further comprising:
applying pressure to said plurality of EEG sensors to force said EEG sensors against the exterior surface of said Patient's head.

20. The method of collecting EEG sensor data of claim 19, further comprising:
providing movement and/or rotation of said EEG sensors to enhance contact between said EEG sensors and the scalp of said Patient.
